SEQUENCE LISTING (38 sequences)

SEQ ID NO: 1 - protein, 12 aa, Homo sapiens
CASRVGIAEAFF

SEQ ID NO: 2 - protein, 13 aa, Homo sapiens
CASSEDSNQPQHF

SEQ ID NO: 3 - protein, 14 aa, Homo sapiens
CASSLGTGYSPLHF

SEQ ID NO: 4 - protein, 16 aa, Homo sapiens
CASSEHRGRGNQPQHF

SEQ ID NO: 5 - protein, 11 aa, Homo sapiens
CATSNRGIQYF

SEQ ID NO: 6 - protein, 15 aa, Homo sapiens
CASSLGDSIYNEQFF

SEQ ID NO: 7 - protein, 14 aa, Homo sapiens
CASSSGEANYGYTF

SEQ ID NO: 8 - protein, 15 aa, Homo sapiens
CASSEWVGGNSPLHF

SEQ ID NO: 9 - protein, 12 aa, Homo sapiens
CASSQESYEQYF

SEQ ID NO: 10 - protein, 15 aa, Homo sapiens
CASSRDIGLSQPQHF

SEQ ID NO: 11 - protein, 15 aa, Homo sapiens
CASSESRGVNGELFF

SEQ ID NO: 12 - protein, 20 aa, Homo sapiens
CASSIGGGTSGRAGYNEQFF

SEQ ID NO: 13 - protein, 12 aa, Homo sapiens
CSAQGPHYGYTF

SEQ ID NO: 14 - protein, 16 aa, Homo sapiens
CASSPPRDYSGNTIYF

SEQ ID NO: 15 - protein, 13 aa, Homo sapiens
CASSRNRNTEAFF

SEQ ID NO: 16 - protein, 17 aa, Homo sapiens
CASSVEGGLGSEQPQHF

SEQ ID NO: 17 - protein, 14 aa, Homo sapiens
CASTQGGRGGEQYF

SEQ ID NO: 18 - protein, 16 aa, Homo sapiens
CSASIRTADRAEKLFF

SEQ ID NO: 19 - protein, 10 aa, Homo sapiens
DEGGWACLVY

SEQ ID NO: 20 - protein, 9 aa, Homo sapiens
MADQLVAVI

SEQ ID NO: 21 - protein, 10 aa, Homo sapiens
VLYSNRFAAY

SEQ ID NO: 22 - protein, 10 aa, Homo sapiens
YSNRFAAYAK

SEQ ID NO: 23 - protein, 9 aa, Homo sapiens
SATMSGVTI

SEQ ID NO: 24 - protein, 10 aa, Homo sapiens
STPICSSRRK

SEQ ID NO: 25 - protein, 9 aa, Homo sapiens
EEVLHTMPI

SEQ ID NO: 26 - protein, 9 aa, Homo sapiens
SISSGESIK

SEQ ID NO: 27 - protein, 11 aa, Homo sapiens
LVYKEKLIIWK

SEQ ID NO: 28 - protein, 9 aa, Homo sapiens
GSQVRYACK

SEQ ID NO: 29 - protein, 9 aa, Homo sapiens
LEDNPESTV

SEQ ID NO: 30 - protein, 9 aa, Homo sapiens
SIKVLGTEK

SEQ ID NO: 31 - protein, 10 aa, Homo sapiens
KESQPALELK

SEQ ID NO: 32 - protein, 9 aa, Homo sapiens
KAHLIRPRK

SEQ ID NO: 33 - protein, 9 aa, Homo sapiens
YVMASVASV

SEQ ID NO: 34 - protein, 9 aa, Homo sapiens
DEAYVMASV

SEQ ID NO: 35 - protein, 10 aa, Homo sapiens
KEILDEAYVM

SEQ ID NO: 36 - protein, 10 aa, Homo sapiens
SSQPSPSDPK

SEQ ID NO: 37 - protein, 9 aa, Homo sapiens
SQAAVGPQK

SEQ ID NO: 38 - protein, 10 aa, Homo sapiens
YLSFIKILLK

(19) United States
(12) Patent Application Publication
(10) Pub. No.: US 2022/0257735 A1
(43) Pub. Date: Aug. 18, 2022

(54) A PEPTIDE-BASED SCREENING METHOD TO IDENTIFY NEOANTIGENS FOR USE WITH TUMOR INFILTRATING LYMPHOCYTES

(71) Applicant: H. LEE MOFFITT CANCER CENTER AND RESEARCH INSTITUTE, INC., Tampa, FL (US)

(72) Inventors: Benjamin C. CREELAN, Tampa, FL (US); Eric B. HAURA, St. Pete Beach, FL (US); Scott ANTONIA, Durham, NC (US); Chao WANG, Tampa, FL (US)

(21) Appl. No.: 17/622,375

(22) PCT Filed: Jun. 24, 2020
(86) PCT No.: PCT/US2020/039276

§ 371 Date: Dec. 23, 2021

Related U.S. Application Data
(60) Provisional application No. 62/865,697, filed on Jun. 24, 2019.
(60) Provisional application No. 62/979,386, filed on Feb. 20, 2020.

(51) Int. Cl. A61K 39/00; G01N 33/50

(52) U.S. Cl. CPC A61K 39/0011; A61K 2039/5158; G01N 33/5011; G01N 33/505

(57) ABSTRACT

Disclosed are methods for identifying neoantigens and methods of treating cancer using neoantigens identified by said methods. The disclosure herein provide for methods for identifying neoantigens that can be used as a target for the treatment of a cancer, immunize a subject against a cancer, stimulate/induce immune responses, and/or isolate T cells that are reactive to said neoantigens.

This application claims the benefit of U.S. Provisional Application No. 62/979,386, filed on Feb. 20, 2020 and U.S. Provisional Application No. 62/865,697, filed on Jun. 24, 2019, applications which are incorporated herein by reference in their entirety.

### BACKGROUND

Neoantigens are antigens created by non-synonymous somatic mutations and recognized by unique TCR clonotypes of CD8 or CD4. Studies into neoantigens have shown that some neoantigens that have been able to be identified may induce the durable remissions >1 decade via adoptive cell transfer. Though the therapeutic outlook of neoantigens is promising, multiplex screening methods to identify cancer neoantigens remain challenging. A single tumor has hundreds of potential neoantigens and not all predicted neoantigens are truly epitopes. Moreover, pMHC II binding affinity is unknown and/or incomplete. Lastly, existing methods are limited by expense and scalability. Thus, what are needed are convenient and economic methods to identify relevant neoantigens for clinical applications is urgently needed.

### SUMMARY

Disclosed are methods related to the identification and use of neoantigens.

In one aspect, disclosed herein are methods of screening for neoantigens, the method comprising: a) obtaining a cancerous tissue sample from a subject with a cancer; b) fragmenting a first portion of the tissue sample and culturing said first portion; c) expanding tumor infiltrating lymphocytes (TILs) in the cultured first portion; d) subjecting a second portion of the tissue sample to sequencing (such as, for example whole exosome sequencing or RNA sequencing); e) applying bioinformatics to the sequence data to identify putative neoantigens; co-culturing the putative neoantigens with the expanded TILs; and g) assaying the co-cultured TILs for reactivity to cancer cells from the subject (for example, assaying for reactivity wherein the reactivity is determined by ELISA, ELISpot, and/or TCRVβ sequencing); wherein reactive TILs indicate that the putative neoantigen co-cultured with the TILs is a neoantigen.

Also disclosed herein are methods of screening for neoantigens of any preceding aspect, further comprising obtaining peripheral blood mononuclear cells (PBMCs) from the subject with the cancer. T cells can be isolated from the PBMC from the subject using cell sorting techniques known in the art, including but not limited to magnetic cell sorting (MACS) or fluorescence acquired cell sorting (FACS).

In one aspect, disclosed herein are methods of screening for neoantigens of any preceding aspect, wherein the isolated T cells are co-cultured with the putative neoantigens of step e and assayed for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

Also disclosed herein are method of screening for neoantigens, the method comprising: a) obtaining a cancerous tissue sample from a subject with a cancer; b) obtaining a peripheral blood mononuclear cells (PBMCs) from the subject with the cancer; c) subjecting the cancerous tissue sample to sequencing (such as, for example whole exosome sequencing or RNA sequencing); d) applying bioinformatics to the sequence data to identify putative neoantigens; e) isolating T cells from the PBMC from the subject (isolating T cells from the PBMC using any technique known in the art including, but not limited to magnetic cell sorting MACS or FACS); co-culturing the putative neoantigens with isolated T cells; and g) assaying the co-cultured isolated T cells for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

In one aspect, disclosed herein are neoantigens identified by the disclosed methods. In one aspect, the neoantigens comprise the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38).

In one aspect, disclosed herein are methods of treating, inhibiting, reducing, decreasing, ameliorating, and/or preventing a cancer and/or metastasis in a subject comprising a) obtaining a cancerous tissue sample from the subject with the cancer; b) fragmenting a first portion of the tissue sample and culturing said first portion; c) expanding tumor infiltrating lymphocytes (TILs) in the cultured first portion; d) subjecting a second portion of the tissue sample to sequencing (such as, for example whole exosome sequencing or RNA sequencing); e) applying bioinformatics to the sequence data to identify putative neoantigens; f) co-culturing the putative neoantigens with the expanded TILs; g) assaying the co-cultured TILs for reactivity to cancer cells from the subject (for example, assaying for reactivity wherein the reactivity is determined by ELISA, ELISpot, and/or TCRVβ sequencing); wherein reactive TILs indicate that the putative neoantigen co-cultured with the TILs is a neoantigen; h) isolating, culturing, and expanding TILs that are reactive to the neoantigen; i) administering to the subject with the cancer an anti-cancer therapeutic agent; j) measuring the clinical benefit of the treatment; and k) administering TILs specific for a neoantigen to the subject when there is no or minimal clinically relevant benefit from the administration of the anti-cancer therapeutic agent.

Also disclosed herein are methods of treating, inhibiting, reducing, decreasing, ameliorating, and/or preventing a cancer and/or metastasis of any preceding aspect, further comprising obtaining peripheral blood mononuclear cells (PBMCs) from the subject with the cancer. T cells can be isolated from the PBMC from the subject using cell sorting techniques known in the art, including but not limited to magnetic cell sorting (MACS) or fluorescence acquired cell sorting (FACS).

In one aspect, disclosed herein are methods of treating, inhibiting, reducing, decreasing, ameliorating, and/or preventing a cancer and/or metastasis of any preceding aspect, wherein the isolated T cells are co-cultured with the putative neoantigens of step e and assayed for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

In one aspect, it is understood and herein contemplated that steps i) and j) of any preceding method of treatment can be performed at any time prior to step k) including before or after any of steps b), c), d), e), f), g), and/or h).

It is understood and herein contemplated that the neoantigens disclosed herein can be used in the methods of treatment of cancer disclosed herein. For example, the neoantigens can be administered to a subject to stimulate or induce an in vivo response to the tumor by endogenous immune cells such as TILs or administered concurrently with TILs. Alternatively, the neoantigens can be used to screen for TILs reactive to the neoantigen and once identified, said TILs can be expanded (in the presence of the neoantigens) and administered to a patient with a cancer. Thus, in one aspect, disclosed herein are methods of treating, inhibiting, reducing, decreasing, ameliorating, and/or preventing a cancer and/or metastasis of any preceding aspect, comprising administering to a subject with a cancer one or more of the neoantigens comprising the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38) or any other neoantigen identified by the disclosed methods.

Also disclosed herein are methods of stimulating and or inducing an immune response to a cancer comprising administering to a subject with a cancer one or more of the neoantigens comprising the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38) or any other neoantigen identified by the disclosed methods. In one aspect, the method can further comprise administering neoantigen reactive TILs in combination with any of the disclosed neoantigens or any neoantigen identified with the by the disclosed methods. It is understood and herein contemplated that the neoantigens and TILs can be administered in the same formulation, or separately. When administered separately, the TILs and neoantigen can be administered concurrently or 1, 2, 3, 4,5 6, 7, 8, 9, 10, 11, 12, 13, 14, 15, 20, 25, 30, 35, 40, 45, 50, 55, 60, 65, 70, 75, 80, 85, 90, 120 min, 3, 4, 5, 6, 7, 8, 9, 10, 11, 12, 13, 14, 15, 16, 17, 18, 19, 20, 21, 22, 23, 24, 28, 30, 36, 42, 48 hours, 3, 4, 5, 6, 7, 8, 9, 10, 11, 12, 13, 14 days apart with either administration preceding the other.

Also disclosed herein are methods of treating, inhibiting, reducing, decreasing, ameliorating, and/or preventing a cancer and/or metastasis in a subject of any preceding aspect comprising a) obtaining a tissue sample from a subject with a cancer; b) fragmenting a the tissue sample and culturing said fragmented tissue; c) expanding tumor infiltrating lymphocytes (TILs); screening the expanded TILs for TILs reactive to one or more of the neoantigens comprising the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), and/or YLSFIKILLK (SEQ ID NO: 38) or any other neoantigen identified by the methods disclosed herein; administering to the subject TILs that are reactive to one or more neoantigens. In one aspect the reactive TILs can be cultured and expanded prior to administration to the subject. In one aspect, the culturing and expansion of TILs can occur in the presence of the neoantigen.

In one aspect, it is understood that once neoantigens are identified (such as through the disclosed methods), further screening of neoantigens or neoantigen reactive TILs is not required for the expansion of neoantigen reactive TILs as said TILs can simply be expanded from a bulk population in culture by expanding the TILs in the presence of the neoantigen. Thus, in one aspect, disclosed herein are methods of expanding neoantigen reactive TILs comprising obtaining TILs from a subject and culturing the TILs in the presence of any of the neoantigens disclosed herein including but not limited to CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), and/or YLSFIKILLK (SEQ ID NO: 38) or any other neoantigen identified by the methods disclosed herein.

In one aspect, disclosed herein are methods of vaccinating a subject against a cancer comprising administering to a subject one or more neoantigens identified by the method of any preceding aspect (such as, for example, CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38)). For example, disclosed herein are methods of vaccinating a subject against a cancer comprising: a) obtaining a cancerous tissue sample from a subject with a cancer; b) fragmenting a first portion of the tissue sample and culturing said first portion; c) expanding tumor infiltrating lymphocytes (TILs) in the cultured first portion; d) subjecting a second portion of the tissue sample to sequencing; e) applying bioinformatics to the sequence data to identify putative neoantigens; co-culturing the putative neoantigens with the expanded TILs; g) assaying the co-cultured TILs for reactivity to cancer cells from the subject; wherein reactive TILs indicate that the putative neoantigen co-cultured with the TILs is a neoantigen; and h) administering to a subject one or more neoantigens. It is understood and herein contemplated that the vaccine can be administered therapeutically or prophylactically.

In one aspect, disclosed herein are methods of isolating, purifying and/or expanding a TIL population specific for a neoantigen comprising contacting a heterologous TIL population with one or more of the neoantigens disclosed herein and culturing the TILs in the presence of the neoantigen.

Also disclosed herein are methods of vaccinating a subject against a cancer of any preceding aspect, wherein the neoantigens are administered to the subject after initiation of TIL immunotherapy.

In one aspect, disclosed herein are methods of treating a cancer of any preceding aspect further comprising the administration of an anti-cancer therapeutic agent.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic representation of a neoantigen identification flowchart for a peptide-based screening method to identify neoantigen in NSCLC patients.

FIG. 2A shows positive control for peptide-based antigen screening method using known viral peptides. (n=3). Shown are mean±SD, p-value is calculated by repeated measures ANOVA with Dunnett's procedure to control for multiple comparisons. “Fr” denotes tumor fragment number. IFN; interferon.

FIG. 2B shows validation of TIL fragments' reactivity to autologous tumor cells. (n=3 ELISA reaction/bar.). Shown are mean ±SD, p-value is calculated by repeated measures ANOVA with Dunnett's procedure to control for multiple comparisons. “Fr” denotes tumor fragment number. IFN; interferon.

FIG. 3 shows a schematic of the process of tissue resection, TIL infusion and tumor recurrence for a cancer patient.

FIG. 4 shows neoantigen screening using ELISA. Bars indicate mean ±SD. Shown p-value calculated by repeated measures ANOVA with Dunnett's multiple comparison test. Somatic mutated residue(s) shown in bold.

FIG. 5 shows ELISpot assay confirmation of reactivity for Pep#1 (DEGGWACLVY). Bars indicate mean ±SD. Shown p-value calculated by repeated measures ANOVA with Dunnett's multiple comparison test. Somatic mutated residue(s) shown in bold.

FIG. 6 shows a schematic for TCRVβ sequencing.

FIG. 7 shows identification and expansion of neotantigen specific TCRVβ clonotypes.

FIG. 8 shows MANAFEST+ data for various clonotypes.

FIG. 9 shows all MANAFEST+ TCRVβ clonotypes.

FIG. 10 shows the tracking of TCRVβ after TIL infusion.

FIGS. 11A, 11B, 11C, 11D, and 11E show peptide neoantigen screening of Patient 3. FIG. 11A shows a schematic of the process of tissue resection, TIL infusion and tumor recurrence for a cancer patient as shown in FIG. 3, but now providing results of testing for identified putative neoantigens. FIG. 11B shows PBMCs 4 weeks post-TIL (n=2 each). FIG. 11C shows PBMC with new lesion 330 days later. FIG. 11D shows TIL from original tumor (n=3 each). FIG. 11E shows TIL cultured from tumor at progression (n=2 each).

FIG. 12 shows the identification of neoantigens driving T cell responses. Bars indicate mean±SD. Shown is 2-sided p-value calculated by repeated measures ANOVA with Dunnett's multiple comparison test. n=3. APC, antigen-presenting cell; MHC I, major histocompatibility complex class I; Tm; autologous tumor cells.

FIG. 13 shows dynamics of neoantigen-specific T cells over time.

FIG. 14 shows that infused T cells can recognize multiple antigen types. Shown is representative for experiments to date and is not the final dataset. Additional data is forthcoming for more cell samples and more antigens tested from shown patients, and more patients total. SFC, spot-forming colonies. IFN, interferon. neoAg, neoantigen. CT, cancer testis antigen.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

In this specification and in the claims that follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

A “binding molecule” or “antigen binding molecule” (e.g., an antibody or antigen-binding fragment thereof) as provided herein refers in its broadest sense to a molecule that specifically binds an antigenic determinant. In one embodiment, the binding molecule specifically binds to an immunoregulator molecule (such as for example, a transmembrane SEMA4D (CD100) polypeptide of about 150 kDa or a soluble SEMA4D polypeptide of about 120 kDa). In another embodiment, a binding molecule is an antibody or an antigen binding fragment thereof, e.g., MAb 67 or pepinemab.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Neoantigens and Methods of their Use

The disclosure herein provide for methods for identifying neoantigens that can be used as a target for the treatment of a cancer, immunize a subject against a cancer, stimulate/induce immune responses, and/or isolate T cells that are reactive to said neoantigens. Accordingly, in one aspect, disclosed herein are methods of screening for neoantigens, the method comprising: a) obtaining a cancerous tissue sample from a subject with a cancer; b) fragmenting a first portion of the tissue sample and culturing said first portion; c) expanding tumor infiltrating lymphocytes (TILs) in the cultured first portion; d) subjecting a second portion of the tissue sample to sequencing (such as, for example whole exosome sequencing or RNA sequencing); e) applying bioinformatics to the sequence data to identify putative neoantigens; co-culturing the putative neoantigens with the expanded TILs; and g) assaying the co-cultured TILs for reactivity to cancer cells from the subject (for example, assaying for reactivity wherein the reactivity is determined by ELISA, ELISpot, and/or TCRVβ sequencing); wherein reactive TILs indicate that the putative neoantigen co-cultured with the TILs is a neoantigen. It is understood and herein contemplated that the disclosed screening methods can use T cells obtained directly from a subject receiving TIL immunotherapy or from another source. In one aspect, the methods can further comprise obtaining said T cells. Thus, also disclosed herein are methods of screening for neoantigens further comprising obtaining peripheral blood mononuclear cells (PBMCs) from the subject with the cancer. T cells can be isolated from the PBMC from the subject using cell sorting techniques known in the art, including but not limited to magnetic cell sorting (MACS) or fluorescence acquired cell sorting (FACS).

It is understood that the disclosed neoantigens can be identified by taking advantage of the immunological response of T cells from the donor source (e.g., a subject undergoing T cell immunotherapy). Any immunological method disclosed herein is sufficient for this purpose. In one aspect, disclosed herein are methods of screening for neoantigens wherein the isolated T cells are co-cultured with the putative neoantigens of step e and assayed for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

### 1. Immunoassays and Immunological Markers

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis- BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; CyS™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (Di1C18(5)); DIDS; Dihydorhodamine 123 (DHR); Dil (DilC18(3)); I Dinitrophenol; DiO (DiOC18(3)); DiR; DiR (Di1C18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1;YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-genrating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log10MW for known samples, and read off the logMr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P.H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately, and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Biolnvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

In one aspect, disclosed herein are method of screening for neoantigens, the method comprising: a) obtaining a cancerous tissue sample from a subject with a cancer; b) obtaining a peripheral blood mononuclear cells (PBMCs) from the subject with the cancer; c) subjecting the cancerous tissue sample to sequencing (such as, for example whole exosome sequencing or RNA sequencing); d) applying bioinformatics to the sequence data to identify putative neoantigens; e) isolating T cells from the PBMC from the subject (isolating T cells from the PBMC using any technique known in the art including, but not limited to magnetic cell sorting MACS or FACS); co-culturing the putative neoantigens with isolated T cells; and g) assaying the co-cultured isolated T cells for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

It is understood and herein contemplated that the disclosed screening methods result in the identification of neoantigens. Thus, in one aspect, disclosed herein are neoantigens identified by the disclosed methods. In one aspect, the neoantigens comprise the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38).

It is understood and herein contemplated that T cells (such as, for example, TILs) that are reactive to the neoantigens disclosed herein can be administered to a subject with a cancer as a treatment for said cancer. Thus, while the disclosed screening methods are designed to identify neoantigens, once identified, the neoantigens can be used to screen for TILs reactive to the neoantigen and once identified, said TILs can be expanded (in the presence of the neoantigens) and administered to a patient with a cancer. In one aspect, disclosed herein are methods of treating, inhibiting, reducing, decreasing, ameliorating, and/or preventing a cancer and/or metastasis in a subject comprising a) obtaining a cancerous tissue sample from the subject with the cancer; b) fragmenting a first portion of the tissue sample and culturing said first portion; c) expanding tumor infiltrating lymphocytes (TILs) in the cultured first portion; d) subjecting a second portion of the tissue sample to sequencing (such as, for example whole exosome sequencing or RNA sequencing); e) applying bioinformatics to the sequence data to identify putative neoantigens; co-culturing the putative neoantigens with the expanded TILs; g) assaying the co-cultured TILs for reactivity to cancer cells from the subject (for example, assaying for reactivity wherein the reactivity is determined by ELISA, ELISpot, and/or TCRVβ sequencing); wherein reactive TILs indicate that the putative neoantigen co-cultured with the TILs is a neoantigen; h) isolating, culturing, and expanding TILs that are reactive to the neoantigen (neoantigens including, but not limited to CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38)); k) and administering TILs specific for a neoantigen to the subject. In some aspects the treatment methods can comprise i) administering to the subject with the cancer an anti-cancer therapeutic agent and j) measuring the clinical benefit of the treatment prior to administration of TILs specific for a neoantigen, and only administering neoantigen specific TILs when there is no or minimal clinically relevant benefit from the administration of the anti-cancer therapeutic agent alone. It is understood and herein contemplated that steps i) and j) can be performed at any time prior to step k) including before or after any of steps b), c), d), e), f), g), and/or h).

It is understood and herein contemplated that the disclosed cancer treatment, inhibition, reduction, amelioration and/or prevention methods can use T cells obtained directly from a subject receiving TIL immunotherapy or from another source. In one aspect, the methods can further comprise obtaining said T cells. Thus, also disclosed herein are methods of treating, inhibiting, reducing, decreasing, ameliorating, and/or preventing a cancer and/or metastasis further comprising obtaining peripheral blood mononuclear cells (PBMCs) from the subject with the cancer. T cells can be isolated from the PBMC from the subject using cell sorting techniques known in the art, including but not limited to magnetic cell sorting (MACS) or fluorescence acquired cell sorting (FACS).

In one aspect, disclosed herein are methods of treating, inhibiting, reducing, decreasing, ameliorating, and/or preventing a cancer and/or metastasis wherein the isolated T cells are co-cultured with the putative neoantigens of step e and assayed for reactivity to cancer cells from the subject (for example, assaying for reactivity wherein the reactivity is determined by ELISA, ELISpot, and/or TCRVβ sequencing); wherein reactive T cells indicate that the putative neoantigen co-cultured with the T cells is a neoantigen.

It is understood and herein contemplated that the neoantigens disclosed herein can be used in the methods of treatment of cancer disclosed herein. For example, the neoantigens can be administered to a subject to stimulate or induce an in vivo response to the tumor by endogenous immune cells such as TILs or administered concurrently with TILs. Thus, in one aspect, disclosed herein are methods of treating, inhibiting, reducing, decreasing, ameliorating, and/or preventing a cancer and/or metastasis comprising administering to a subject with a cancer one or more of the neoantigens comprising the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38) or any other neoantigen identified by the disclosed methods. For example, disclosed herein are methods of treating, inhibiting, reducing, decreasing, ameliorating, and/or preventing a cancer and/or metastasis comprising a) obtaining a tissue sample from a subject with a cancer; b) fragmenting a the tissue sample and culturing said fragmented tissue; c) expanding tumor infiltrating lymphocytes (TILs); screening the expanded TILs for TILs reactive to one or more of the neoantigens comprising the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), and/or YLSFIKILLK (SEQ ID NO: 38) or any other neoantigen identified by the methods disclosed herein; administering to the subject TILs that are reactive to one or more neoantigens. In one aspect the reactive TILs can be cultured and expanded prior to administration to the subject. In one aspect, the culturing and expansion of TILs can occur in the presence of the neoantigen.

As noted above, by administering a neoantigen to a subject with a cancer or at risk for developing a cancer, the neoantigen is inducing and/or stimulating an endogenous immune response to the neoantigen in the subject. That is, the subject is being vaccinated (therapeutically or prophylactically) against the cancer using the neoantigen. Thus, in one aspect, disclosed herein are methods of vaccinating a subject against a cancer and/or stimulating and/or inducing an immune response to a cancer in a subject comprising administering to a subject with a cancer one or more of the neoantigens comprising the amino acid sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38) or any other neoantigen identified by the disclosed methods. For example, disclosed herein are methods of vaccinating a subject against a cancer and/or inducing and/or stimulating a response to a cancer in a subject or likely to develop in a subject, said method comprising: a) obtaining a cancerous tissue sample from a subject with a cancer; b) fragmenting a first portion of the tissue sample and culturing said first portion; c) expanding tumor infiltrating lymphocytes (TILs) in the cultured first portion; d) subjecting a second portion of the tissue sample to sequencing; e) applying bioinformatics to the sequence data to identify putative neoantigens; f) co-culturing the putative neoantigens with the expanded TILs; g) assaying the co-cultured TILs for reactivity to cancer cells from the subject; wherein reactive TILs indicate that the putative neoantigen co-cultured with the TILs is a neoantigen; and h) administering to a subject one or more neoantigens. It is understood and herein contemplated that the vaccine can be administered therapeutically or prophylactically.

In one aspect, the methods of treating, inhibiting, reducing, decreasing, ameliorating, and/or preventing a cancer and/or metastasis can further comprise administering neoantigen reactive TILs in combination with any of the disclosed neoantigens or any neoantigen identified with the by the disclosed methods. It is understood and herein contemplated that the neoantigens and TILs can be administered in the same formulation, or separately. When administered separately, the TILs and neoantigen can be administered concurrently or 1, 2, 3, 4,5 6, 7, 8, 9, 10, 11, 12, 13, 14, 15, 20, 25, 30, 35, 40, 45, 50, 55, 60, 65, 70, 75, 80, 85, 90, 120 min, 3, 4, 5, 6, 7, 8, 9, 10, 11, 12, 13, 14, 15, 16, 17, 18, 19, 20, 21, 22, 23, 24, 28, 30, 36, 42, 48 hours, 3, 4, 5, 6, 7, 8, 9, 10, 11, 12, 13, 14 days apart with either administration preceding the other.

The disclosed methods and any neoantigen disclosed herein can be used to treat, inhibit, reduce, decrease, ameliorate, and/or prevent any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, it is understood the treatment of cancer does not need to be limited to the administration of neoantigens and/or neoantigen-specific T cells, but can include the further administration of anti-cancer agents to treat, inhibit, reduce, decrease, ameliorate, and/or prevent a cancer or metastasis. Anti-cancer therapeutic agents (such as chemotherapeutics, immunotoxins, peptides, and antibodies) that can be used in the methods of treating, inhibiting, reducing, decreasing, ameliorating, and/or preventing a cancer and/or metastasis and in combination with any of the disclosed neoantigens or any CART cells, TIL, or MIL specific for said neoantigen can comprise any anti-cancer therapeutic agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil--Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil--Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil--Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

In one aspect, it is understood that once neoantigens are identified (such as through the disclosed methods), further screening of neoantigens or neoantigen reactive TILs is not required for the expansion of neoantigen reactive TILs as said TILs can simply be expanded from a bulk population in culture by expanding the TILs in the presence of the neoantigen. Thus, in one aspect, disclosed herein are methods of expanding neoantigen reactive TILs comprising obtaining TILs from a subject and culturing the TILs in the presence of any of the neoantigens disclosed herein including but not limited to CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), and/or YLSFIKILLK (SEQ ID NO: 38) or any other neoantigen identified by the methods disclosed herein. Thus, in one aspect, disclosed herein are methods of isolating, purifying and/or expanding a TIL population specific for a neoantigen comprising contacting a heterologous TIL population with one or more of the neoantigens disclosed herein and culturing the TILs in the presence of the neoantigen.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

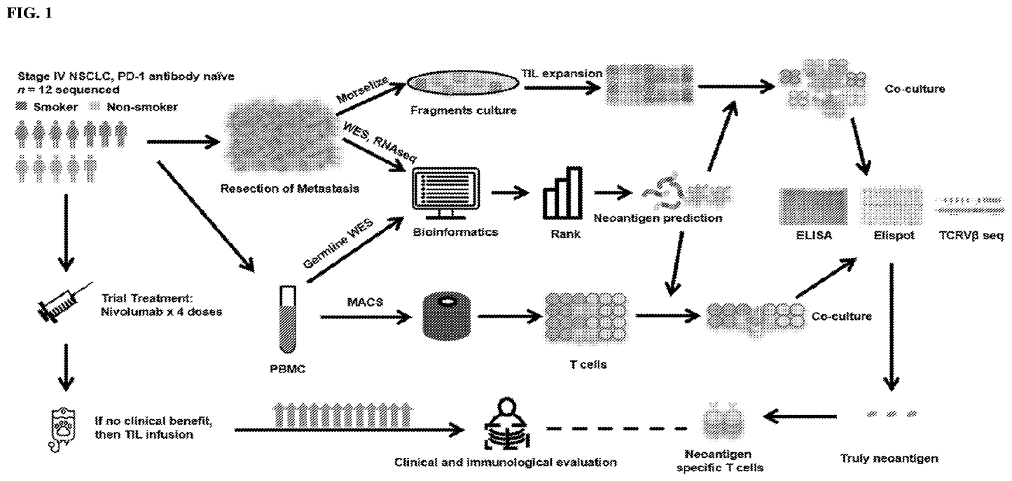

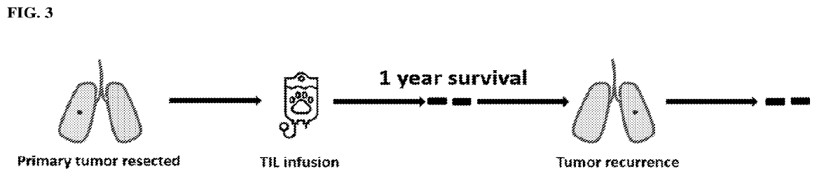

As shown in FIG. 1, this process is developed to identify neoantigens and hen utilize neoantigens to isolate neoantigen specific TILs for the treatment of a cancer. PBMCs and/or tissue resections were collected from patients before and after combination therapy. PBMC were subjected to magnetic cell separation (MACS) to isolate T cells from the PBMC. Concurrently, metastatic tissue resections were either fragmented or subjected to sequence analysis through whole exosome sequencing (WES) or RNAseq analysis. The fragmented sections were cultured to expand tumor infiltrating lymphocytes. Bioinformatic analysis was performed on the sequencing data for the determination of the number and sequence of each of the productive unique VP gene identified within each sample and the degree of clone sharing between samples. FIG. 3 shows a schematic of the process of tissue resection, TIL infusion and tumor recurrence for a cancer patient.

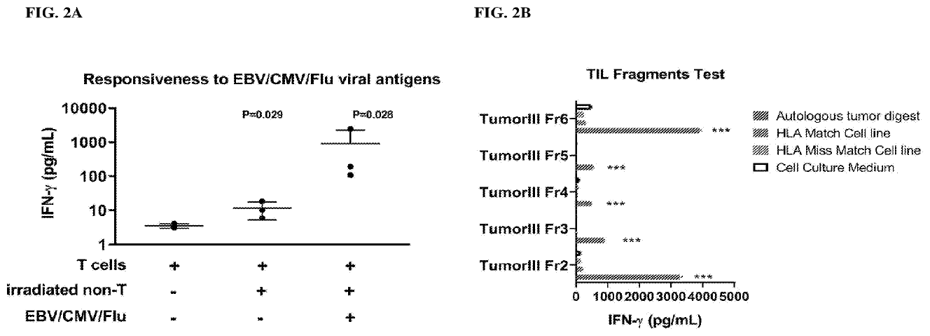

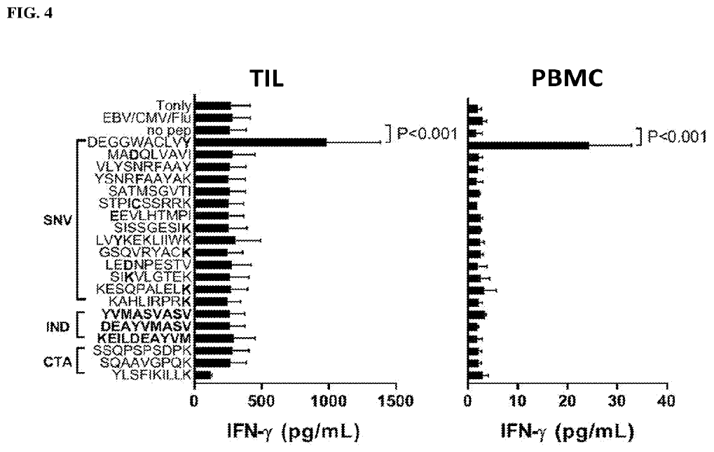

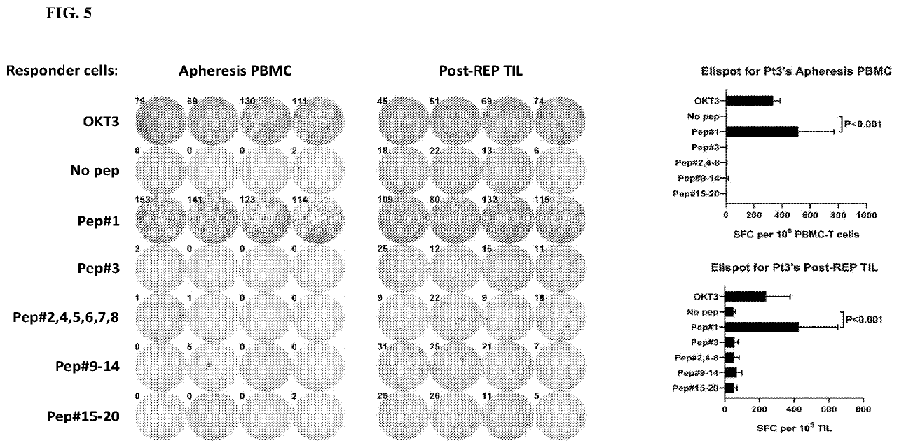

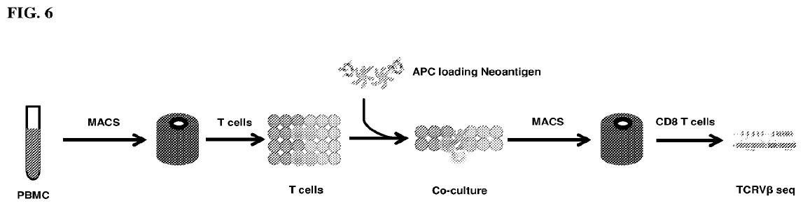

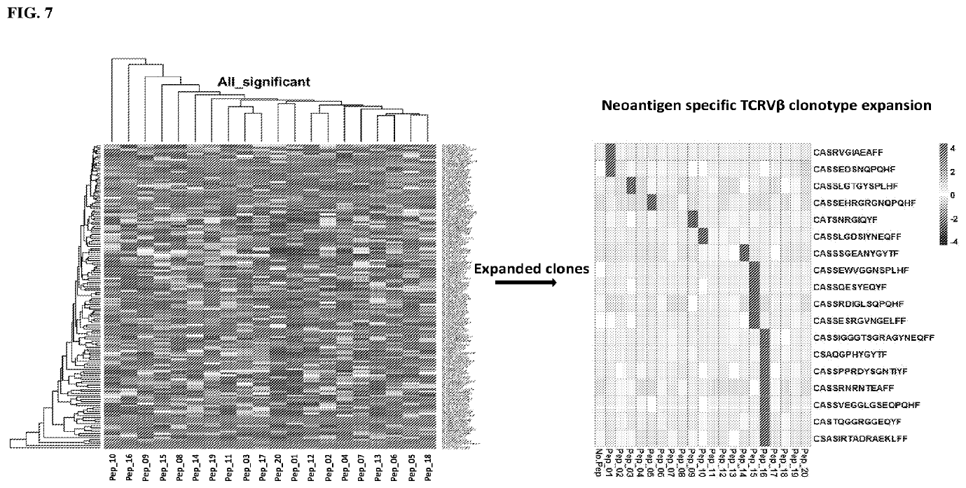

To assay the reactivity of the unique peptides, ELISAs, ELISpots, and TCRVβ sequencing was used. To show the validation of the assays to antigen screening was performed on known viral peptides (FIG. 2A) and TIL fragments (FIG. 2B). Briefly, potential neoantigens were contacted with TILs isolated from either expanded from fragmented tissue resections or isolated via MACS from PBMC and the ELISA, ELISpot, or TVR sequencing performed to show reactivity. (See FIGS. 4, 5, 6, and 7).

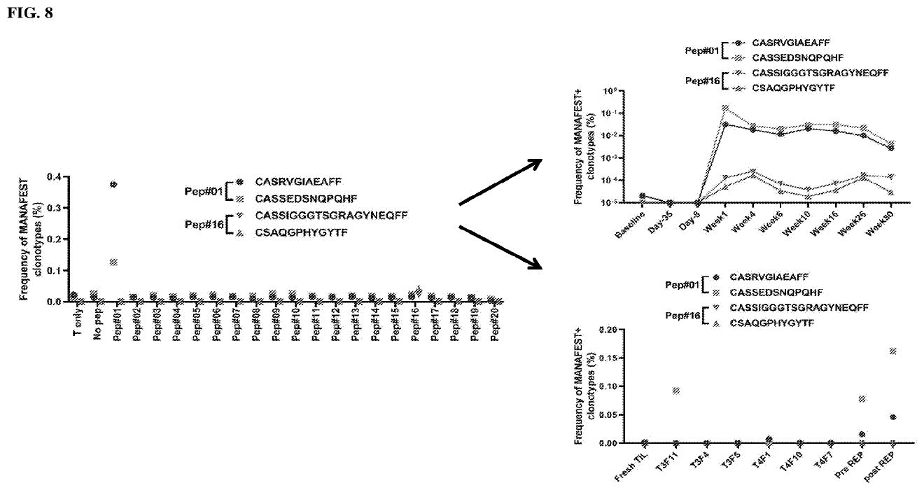

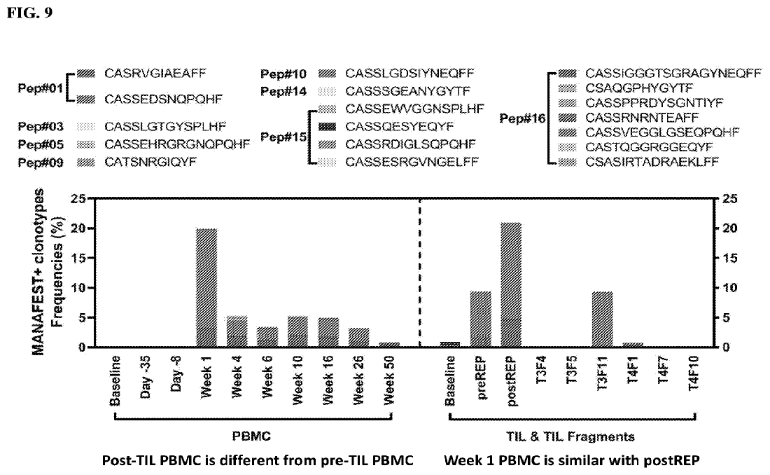

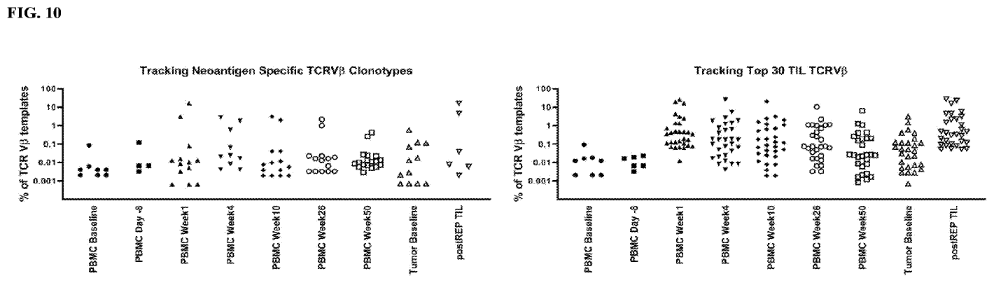

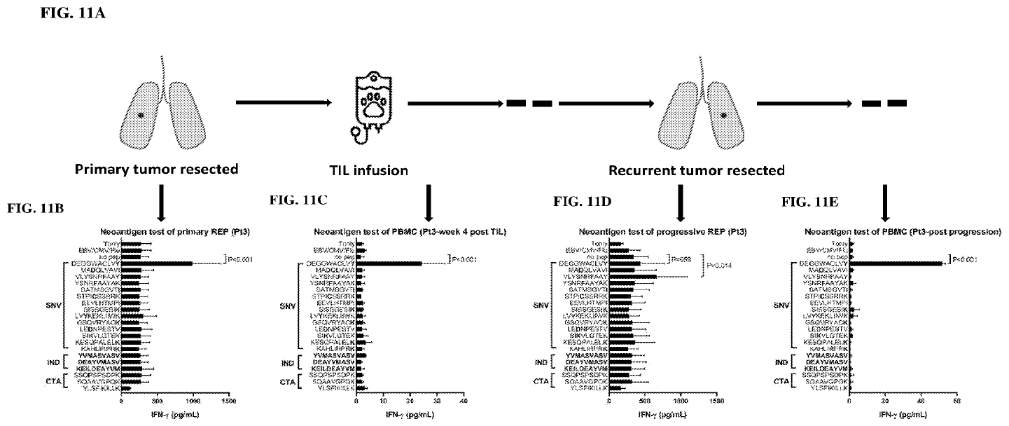

To define the immunogenicity of every individual candidate predicted in the analysis of mutational burden analysis, the presence of reactive T cell clones in peripheral blood were individually quantified in MANAFEST analysis. MANAFEST is a novel, scalable method to evaluate candidate tumor neoantigens for their ability to induce T cell responses (more sensitive and specific than conventional ELISPOT or ELISA). Briefly, up to 30 mutations associated neoantigen candidates per tumor (MANAs) were synthesized (New England Peptide, Inc) for reactivity analysis. The T cell fraction of PBMCs collected before and 3 and 7 weeks after initial treatment for every patient were separated using beads. The non-T cell fraction is then gamma-irradiated and autologous T and irradiated non-T cells are put back together. Predicted neoantigens are then added to specific wells in triplicate, along with cytokines (such as, IL-2, IL-7 and IL-15). On days 3 and 7, half the medium is replaced with fresh medium containing cytokines. CD8 T cells are then separated using a CD4 positive selection kit and subjected to ImmunoSeq analysis. Controls include CEF peptides (epitopes from common viral infections) or no peptide. TCR beta chains expanded >10-fold in response to individual peptides (but not in response to other neoantigens or control CEF peptides) are considered positive. (See FIGS. 8 and 9). FIG. 8 shows MANAFEST+data for various clonotypes. The data reveal that Neoantigen specific clones exist in some of TIL fragments as well as postREP. Additionally, the frequencies of neoantigens increased dramatically after TIL infusion and were very low in baseline tumors (FIG. 10). Additionally, T cell profiles of pre-TIL T cells are different from post-TIL T cells and more neoantigen specific clones emerged after TIL infusion. FIG. 11 shows the results of peptide neoantigen screening of one patient.

Following the experiments provided herein certain conclusions could be made. First, from a detection on procedural basis, peptide-based antigen screening can effectively detect viral peptide controls. Additionally, irradiated non-T cells are preferred as APCs and the supernatant IFN-γ levels are best detected on Day 3 with less background IFN-γ. Also, peptide screening can effectively identify private tumor neoantigens using both PBMCs and TIL. Interestingly, TILs have a higher sensitivity than PBMCs for tumor neoantigen detection and cultured lung cancer TILs were shown to retain autologous tumor recognition.

It is also shown herein that while ELISA and ELISpot are suitable for assays for the identification of neoantigens, TCRVβ sequencing assay is more sensitive. The experiments also show that Pep#01 is a neoantigen for patient #3 (Pt3).

The data provided herein also show that TIL infusion helps increase neoantigen-specific TCR clonotypes. Additionally, TIL abundant TCR clonotypes, including neoantigen specific ones, can be retained in recipient blood (recurrent tumor to be determined) for a long time. As much of this data was done in patients with non-small cell lung carcinoma, the current neoantigen screening approach is feasible in lung cancer patients.

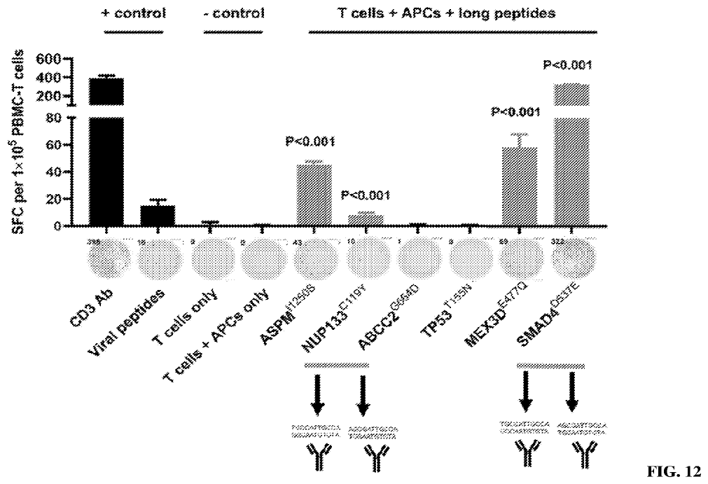

WES and RNA-Seq were performed on the baseline tumor from which TIL was cultured. Custom synthesized peptides corresponding to 9'mers or 25'mers based on the top predicted mutations by expression level and MHC affinity were used. Dendritic cells were obtained from fresh PBMCs and cultured and a small volume apheresis performed at Day +30 after TIL. ELI Spot colony formation was tested after incubation with peptides and autologous dendritic cells. Controls, specifically positive controls were included with viral peptides (CEF) and/or tetanus toxoid. Negative controls include T cells only, and APCs+T cells without peptide. For the positive peptides, we test for selective CDR3 expansion after 10 days of co-culture (MANAFEST). Additionally, TIL were tested for autologous reactivity against a tumor digest (suspension) using ELISA (FIG. 12).

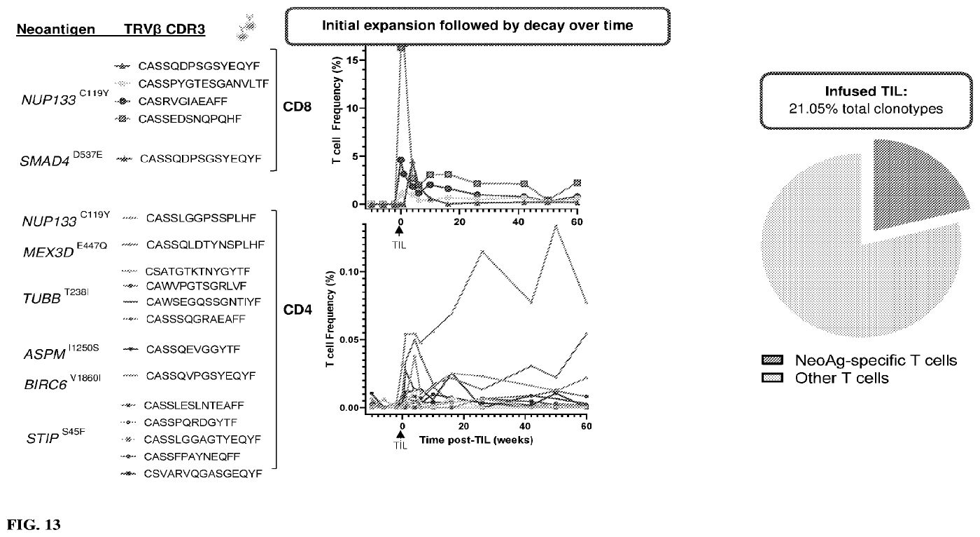

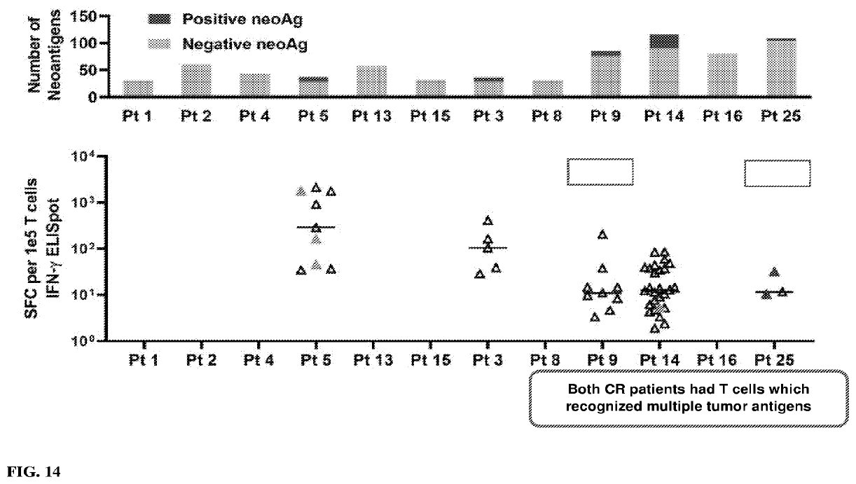

Looking at the dynamics of the response (FIG. 13), note that there is an initial expansion of the neoantigen specific T cell clonotypes relative to other T cells after infusion. Taken altogether, the antigen-specific T cells are 21% of the total TIL infused. It is noteworthy that both complete response patients have had specific neoantigens screen positive from their TIL, including two CT antigens (FIG. 14). There is trend that patients with a clinical benefit seemed to derive more likelihood for neoAg-specific T cells.

What is claimed is:

1. A method of screening for neoantigens, the method comprising:
a) obtaining a cancerous tissue sample from a subject with a cancer;
b) fragmenting a first portion of the tissue sample and culturing said first portion;
c) expanding tumor infiltrating lymphocytes (TILs) in the cultured first portion;
d) subjecting a second portion of the tissue sample to sequencing;
e) applying bioinformatics to the sequence data to identify putative neoantigens;
f) co-culturing the putative neoantigens with the expanded TILs; and
g) assaying the co-cultured TILs for reactivity to cancer cells from the subject;
wherein reactive TILs indicate that the putative neoantigen co-cultured with the TILs is a neoantigen.

2. The method of claim 1, wherein the sequencing applied to the second portion of the tissue sample is whole exosome sequencing or RNA sequencing.

3. The method of claim 1, further comprising obtaining peripheral blood mononuclear cells (PBMCs) from the subject with the cancer.

4. The method of claim 3, further comprising isolating T cells from the PBMC from the subject; wherein T cells are isolated from the PBMCs using magnetic cell sorting (MACS) or fluorescence acquired cell sorting (FACS).

5. The method of claim 4, wherein the isolated T cells are co-cultured with the putative neoantigens of step e and assayed for reactivity to cancer cells from the subject; wherein reactive T cells indicate that the putative neoantigen co-cultured with the T cells is a neoantigen.

6. The method of any of claims 1-5, wherein the reactivity is determined by ELISA, ELISpot, and/or TCRVβ sequencing.

7. A method of screening for neoantigens, the method comprising:
a) obtaining a cancerous tissue sample from a subject with a cancer;
b) obtaining a peripheral blood mononuclear cells (PBMCs) from the subject with the cancer;
c) subjecting the cancerous tissue sample to sequencing;
d) applying bioinformatics to the sequence data to identify putative neoantigens;
e) isolating T cells from the PBMC from the subject; wherein T cells are isolated from the PBMCs using magnetic cell sorting (MACS) or fluorescence acquired cell sorting (FACS);
f) co-culturing the putative neoantigens with isolated T cells; and
g) assaying the co-cultured isolated T cells for reactivity to cancer cells from the subject;
wherein reactive T cells indicate that the putative neoantigen co-cultured with the T cells is a neoantigen.

8. The method of claim 1, wherein the sequencing applied to the second portion of the tissue sample is whole exosome sequencing or RNA sequencing.

9. The method of any of claims 1-5, wherein the reactivity is determined by ELISA, ELISpot, and/or TCRVβ sequencing.

10. A method of treating a subject with a cancer comprising
a) obtaining a cancerous tissue sample from the subject with the cancer;
b) fragmenting a first portion of the tissue sample and culturing said first portion;
c) expanding tumor infiltrating lymphocytes (TILs) in the cultured first portion;
d) subjecting a second portion of the tissue sample to sequencing;
e) applying bioinformatics to the sequence data to identify putative neoantigens;
f) co-culturing the putative neoantigens with the expanded TILs;
g) assaying the co-cultured TILs for reactivity to cancer cells from the subject; wherein reactive TILs indicate that the putative neoantigen co-cultured with the TILs is a neoantigen;
h) isolating, culturing, and expanding TILs that are reactive to the neoantigen;
i) administering to the subject with the cancer an anti-cancer therapeutic agent;
j) measuring the clinical benefit of the treatment; and
k) administering TILs specific for a neoantigen to the subject when there is no or minimal clinically relevant benefit from the administration of the anti-cancer therapeutic agent.

11. The method of claim 10, wherein the sequencing applied to the second portion of the tissue sample is whole exosome sequencing or RNA sequencing.

12. The method of claim 10, further comprising obtaining peripheral blood mononuclear cells (PBMCs) from the subject with the cancer.

13. The method of claim 12, further comprising isolating T cells from the PBMC from the subject; wherein T cells are isolated from the PBMCs using magnetic cell sorting (MACS) or fluorescence acquired cell sorting (FACS).

14. The method of claim 13, wherein the isolated T cells are co-cultured with the putative neoantigens of step e and assayed for reactivity to cancer cells from the subject; wherein reactive T cells indicate that the putative neoantigen co-cultured with the T cells is a neoantigen.

15. The method of any of claims 10-14, wherein the reactivity is determined by ELISA, ELISpot, and/or TCRVβ sequencing.

16. A method of treating a subject with a cancer comprising administering to the subject tumor infiltrating lymphocytes (TILs) to the subject; wherein the TILs are reactive to one or more neoantigens comprising the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38).

17. The method of treating a subject with a cancer of claim 16, wherein the neoantigen is also administered to the subject.

18. The method of treating a subject with a cancer of claim 16, wherein the TILs are expanded in vitro in the presence of one or more of the neoantigens prior to administration of the TILs.

19. The method of treating a subject with a cancer of any of claims 16-18, wherein the TILs and neoantigen are administered in the same formulation.

20. The method of treating a subject with a cancer of any of claims 16-19, wherein the TILs and neoantigen are administered concurrently.

21. The method of treating a subject with a cancer of any of claims 16-20, wherein the TILs are obtained from the subject that is being treated.

22. A method of expanding tumor infiltrating lymphocytes (TILs) comprising obtaining TILs and culturing the TILS in the presence of one or more neoantigens comprising the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38).

23. The method of claim 22, wherein the TILs are obtained from a subject with a cancer.

24. A method of vaccinating a subject against a cancer comprising administering to a subject one or more neoantigens identified by the method of any of claims 1-9.

25. A method of vaccinating a subject against a cancer comprising:
a) obtaining a cancerous tissue sample from a subject with a cancer;
b) fragmenting a first portion of the tissue sample and culturing said first portion;
c) expanding tumor infiltrating lymphocytes (TILs) in the cultured first portion;
d) subjecting a second portion of the tissue sample to sequencing;
e) applying bioinformatics to the sequence data to identify putative neoantigens;
f) co-culturing the putative neoantigens with the expanded TILs;
g) assaying the co-cultured TILs for reactivity to cancer cells from the subject; wherein reactive TILs indicate that the putative neoantigen co-cultured with the TILs is a neoantigen; and
h) administering to a subject one or more neoantigens.

26. The method of claim 24 or 25, wherein the vaccine is administered therapeutically.

27. The method of any of claims 24-26, wherein the one or more neoantigens comprise the sequence the sequence CASRVGIAEAFF (SEQ ID NO: 1), CASSEDSNQPQHF (SEQ ID NO: 2), CASSLGTGYSPLHF (SEQ ID NO: 3), CASSEHRGRGNQPQHF (SEQ ID NO: 4), CATSNRGIQYF (SEQ ID NO: 5), CASSLGDSIYNEQFF (SEQ ID NO: 6), CASSSGEANYGYTF (SEQ ID NO: 7), CASSEWVGGNSPLHF (SEQ ID NO: 8), CASSQESYEQYF (SEQ ID NO: 9), CASSRDIGLSQPQHF (SEQ ID NO: 10), CASSESRGVNGELFF (SEQ ID NO: 11), CASSIGGGTSGRAGYNEQFF (SEQ ID NO: 12), CSAQGPHYGYTF (SEQ ID NO: 13), CASSPPRDYSGNTIYF (SEQ ID NO: 14), CASSRNRNTEAFF (SEQ ID NO: 15), CASSVEGGLGSEQPQHF (SEQ ID NO: 16), CASTQGGRGGEQYF (SEQ ID NO: 17), CSASIRTADRAEKLFF (SEQ ID NO: 18), DEGGWACLVY (SEQ ID NO: 19), MADQLVAVI (SEQ ID NO: 20), VLYSNRFAAY (SEQ ID NO: 21), YSNRFAAYAK (SEQ ID NO: 22), SATMSGVTI (SEQ ID NO: 23), STPICSSRRK (SEQ ID NO: 24), EEVLHTMPI (SEQ ID NO: 25), SISSGESIK (SEQ ID NO: 26), LVYKEKLIIWK (SEQ ID NO: 27), GSQVRYACK (SEQ ID NO: 28), LEDNPESTV (SEQ ID NO: 29), SIKVLGTEK (SEQ ID NO: 30), KESQPALELK (SEQ ID NO: 31), KAHLIRPRK (SEQ ID NO: 32), YVMASVASV (SEQ ID NO: 33), DEAYVMASV (SEQ ID NO: 34), KEILDEAYVM (SEQ ID NO: 35), SSQPSPSDPK (SEQ ID NO: 36), SQAAVGPQK (SEQ ID NO: 37), or YLSFIKILLK (SEQ ID NO: 38).

28. The method of any of claims 24-27, wherein the neoantigens are administered to the subject after initiation of TIL immunotherapy.
